(19) United States
(12) Patent Application Publication
(10) Pub. No.: US 2016/0228297 A1
(43) Pub. Date: Aug. 11, 2016

(54) MULTI-COMPONENT FIBERS, NONWOVEN WEBS, AND ARTICLES COMPRISING A POLYDIORGANOSILOXANE POLYAMIDE

(71) Applicant: 3M INNOVATIVE PROPERTIES COMPANY, Saint Paul, MN (US)

(72) Inventors: Junkang J. Liu, Woodbury, MN (US); Bradley W. Eaton, Woodbury, MN (US); David S. Hays, Woodbury, MN (US)

(21) Appl. No.: 15/024,078

(22) PCT Filed: Sep. 24, 2014
(86) PCT No.: PCT/US2014/057080

§ 371 Date: Mar. 23, 2016

Related U.S. Application Data
(60) Provisional application No. 61/886,690, filed on Oct. 4, 2013.

(51) Int. Cl. A61F 13/02; D01F 8/06

(52) U.S. Cl. CPC A61F 13/0259; D01F 8/06; A61F 2013/00238

(57) ABSTRACT

Nonwoven webs are described wherein the web has an inverted MVTR of at least 10,000 g/m²/24 h and a resistance to water penetration of at least 10 min according to EN-20811. In a favored embodiment, the nonwoven web comprises a multi-component fiber. Also described are multi-component fiber comprising a core and outer layer and nonwoven webs comprising such. At least a portion of the outer layer comprises a first melt processable composition comprising a polydiorganosiloxane polyamide copolymer. The core comprises a second melt processable composition that does not comprise a polydiorganosiloxane polymer. The multi-component fiber comprises 5 to 25 wt-% of the polydiorganosiloxane polyamide copolymer

### SUMMARY

In one embodiment, a nonwoven web is described wherein the web has an inverted MVTR of at least 10,000 g/m²/24 h and a resistance to water penetration of at least 10 min according to EN20811. In a favored embodiment, the nonwoven web comprises a multi-component fiber.

In another embodiment, a multi-component fiber is described comprising a core and outer layer. At least a portion of the outer layer comprises a first melt processable composition comprising a polydiorganosiloxane polyamide copolymer. The core comprises a second melt processable composition that does not comprise a polydiorganosiloxane polymer. The multi-component fiber comprises 5 to 25 wt-% of the polydiorganosiloxane polyamide copolymer.

Also described is a nonwoven web comprises the multi-component fiber described herein. The nonwoven web is suitable for use as a backing of a medical article.

### BRIEF DESCRIPTION OF THE DRAWINGS

The invention may be further illustrated by reference to the accompanying drawings wherein:

FIG. 1 is a top perspective view of one embodiment of a medical dressing;

FIG. 2A is a top perspective view of another embodiment of a medical dressing; and

FIG. 2B is a bottom perspective view of the dressing of FIG. 2A with the liner removed.

### DETAILED DESCRIPTION OF PREFERRED EMBODIMENTS

The terms “polymer” and “polymeric material” refer to both materials prepared from one monomer such as a homopolymer or to materials prepared from two or more monomers such as a copolymer, terpolymer, or the like. “Polymer” also may refer to polymers that have been chemically modified post polymerization such as by grafting and the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The term “polydiorganosiloxane” refers to a polymer comprising a divalent segment of formula

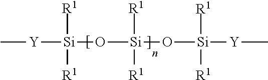

where each R¹ is independently an alkyl, haloalkyl, aralkyl, alkenyl, aryl, or aryl substituted with an alkyl, alkoxy, or halo; each Y is independently an alkylene, aralkylene, or a combination thereof; and subscript n is independently an integer of 40 to 1500. In some embodiments, the polydiorganosiloxane comprises divalent segments in combination with higher valency (e.g. branched) segments such as trivalent or tetravalent segments.

The term “adjacent” means that a first layer is positioned near a second layer. The first layer can contact the second layer or can be separated from the second layer by one or more additional layers.

The terms “room temperature” and “ambient temperature” are used interchangeably to mean a temperature in the range of 20° C. to 25° C.

Unless otherwise indicated, all numbers expressing feature sizes, amounts, and physical properties used in the specification and claims are to be understood as being modified in all instances by the term “about.” Accordingly, unless indicated to the contrary, the numbers set forth are approximations that can vary depending upon the desired properties using the teachings disclosed herein.

The present invention is directed to fibers comprising a polydiorganosiloxane polyamide. The invention will be described herein with respect to a preferred polydiorganosiloxane polyamide, i.e. a polydiorganosiloxane polyoxamide. Such fibers typically have an average diameter of no greater than about 100 μm, and are useful in making nonwoven webs that can be used in making a wide variety of products. Preferably, such fibers have an average diameter of no greater than about 50 μm. Fibers of no greater than about 50 μm are often referred to as “microfibers.” In some embodiments, the microfibers have an average fiber diameter of no greater than 40 μm, or 30 μm, or 25 μm. Improved liquid barrier properties are more typical when the average fiber diameter is less than 20 μm, or 19 μm, or 18 μm, or 17 μm, or 16 μm, or 15 μm, or 14 μm, or 13 μm. In some embodiments, the microfibers have an average diameter of at least 5 μm or 6 μm or 7 μm.

Polydiorganosiloxane polyoxamide copolymers are advantageous because they can possess one or more of the following properties: resistance to ultraviolet light; good thermal and oxidative stability; good permeability to many gases; low surface energy; low index of refraction;

low glass transition temperature; good hydrophobicity; good dielectric properties; and good biocompatibility. Fibers made of such polymers, and nonwoven webs of such fibers, are particularly desirable because they provide a material with a high surface area. The nonwoven webs also have high porosity. Nonwoven webs having a high surface area and porosity are desirable because they possess the characteristics of breathability, moisture transmission, conformability, and good adhesion to irregular surfaces.

Polydiorganosiloxane polyoxamide copolymers are favored over polydiorganosilane polyurea copolymers because of higher thermal stability which can be particularly important for blown microfiber processing. The nonwoven webs described herein generally do not exhibit pressure-sensitive adhesive (PSA) properties at room temperature. Thus, the nonwoven webs described herein typically exhibit (e.g. an initial) 90 degree peel strength to polypropylene of no greater than 25, 20, 15, 10, or 5 grams per 2.54 centimeter width as measured by ASTM D3330-87. The (e.g. initial) peel strength is typically zero.

Suitable polydiorganosiloxane polyamide (e.g. copolymer) compositions are those that are capable of being extruded and forming fibers in a melt process, such as a spunbond process or a melt-blown process, without substantial degradation or gelling. The polydiorganosiloxane polyamide (e.g. copolymer) compositions can be heated to a temperature up to 200° C., up to 225° C., up to 250° C., up to 275° C., or up to 300° C. without noticeable degradation of the material. For example, when heated in a thermogravimetric analyzer in the presence of air, the copolymers often have less than a 10 percent weight loss when scanned at a rate 50° C. per minute in the range of 20° C. to about 350° C. Alternatively, the copolymers can often be heated at a temperature such as 250° C. for 1 hour in air without apparent degradation as determined by no detectable loss of physical properties.

Suitable polydiorganosiloxane polyamide (e.g. copolymer) compositions have a sufficiently low viscosity in the melt such that they can be readily extruded. The polydiorganosiloxane polyamide (e.g. copolymer) composition preferably has a complex viscosity at a temperature in a range from about 275° C. to 325° C. (i.e. at the melt blowing temperature) of at least about 500 poise and typically no greater than 5000 poise at a shear rate of 1 hertz as measured by the test method described in the examples. Without intending to be bound by theory, it is surmised that the actual viscosity (i.e. “apparent viscosity”) during melt blowing is lower than the complex viscosity at 1 hertz due to the high shear forces of the melt blowing process. In some embodiments, the polydiorganosiloxane polyamide (e.g. copolymer) composition are capable of forming a melt stream in a melt blown process that maintains its integrity with few, if any, breaks in the melt stream. Hence, the polydiorganosiloxane polyamide (e.g. copolymer) compositions have an extensional viscosity that allows them to be drawn effectively into fibers. In other embodiments, the second polymer (e.g. within the core) of a multi-component fiber contributes the desired extensional viscosity.

The fibers described herein have sufficient cohesive strength and integrity at their use temperature such that a web formed therefrom maintains its fibrous structure. Sufficient cohesiveness and integrity typically depends on the overall molecular weight of the polydiorganosiloxane polymer, and the concentration and nature of the amide linkages. Fibers comprising suitable polydiorganosiloxane polyamide (e.g. copolymer) compositions typically exhibit relatively low or no cold flow, and display good aging properties, such that the fibers maintain their shape and desired properties (e.g., moisture vapor transmission and liquid barriers properties) over an extended period of time under ambient conditions.

In one favored embodiments, the fibers generally comprise a (e.g. linear) polydiorganosiloxane polyoxamide block copolymer. The block polydiorganosiloxane polyoxamide copolymer contains at least two repeat units of Formula I.

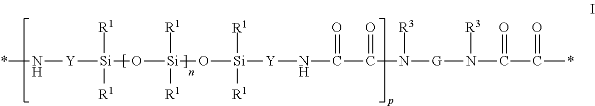

In this formula, each R¹ is independently an alkyl, haloalkyl, aralkyl, alkenyl, aryl, or aryl substituted with an alkyl, alkoxy, or halo, wherein at least 50 percent of the R¹ groups are methyl. Each Y is independently an alkylene, aralkylene, or a combination thereof. Subscript n is independently an integer of 40 to 1500 and the subscript p is an integer of 1 to 10. Group G is a divalent group that is the residue unit that is equal to a diamine of formula R³HN-G-NHR³ minus the two —NHR³ groups. Group R³ is hydrogen or alkyl (e.g., an alkyl having 1 to 10, 1 to 6, or 1 to 4 carbon atoms) or R³ taken together with G and with the nitrogen to which they are both attached forms a heterocyclic group (e.g., R³HN-G-NHR³ is piperazine or the like). Each asterisk (*) indicates a site of attachment of the repeat unit to another group in the copolymer such as, for example, another repeat unit of Formula I.

Suitable alkyl groups for R¹ in Formula I typically have 1 to 10, 1 to 6, or 1 to 4 carbon atoms. Exemplary alkyl groups include, but are not limited to, methyl, ethyl, isopropyl, n-propyl, n-butyl, and iso-butyl. Suitable haloalkyl groups for R¹ often have only a portion of the hydrogen atoms of the corresponding alkyl group replaced with a halogen. Exemplary haloalkyl groups include chloroalkyl and fluoroalkyl groups with 1 to 3 halo atoms and 3 to 10 carbon atoms. Suitable alkenyl groups for R¹ often have 2 to 10 carbon atoms. Exemplary alkenyl groups often have 2 to 8, 2 to 6, or 2 to 4 carbon atoms such as ethenyl, n-propenyl, and n-butenyl. Suitable aryl groups for R¹ often have 6 to 12 carbon atoms. Phenyl is an exemplary aryl group. The aryl group can be unsubstituted or substituted with an alkyl (e.g., an alkyl having 1 to 10 carbon atoms, 1 to 6 carbon atoms, or 1 to 4 carbon atoms), an alkoxy (e.g., an alkoxy having 1 to 10 carbon atoms, 1 to 6 carbon atoms, or 1 to 4 carbon atoms), or halo (e.g., chloro, bromo, or fluoro). Suitable aralkyl groups for R¹ usually have an alkylene group having 1 to 10 carbon atoms and an aryl group having 6 to 12 carbon atoms. In some exemplary aralkyl groups, the aryl group is phenyl and the alkylene group has 1 to 10 carbon atoms, 1 to 6 carbon atoms, or 1 to 4 carbon atoms (i.e., the structure of the aralkyl is alkylene-phenyl where an alkylene is bonded to a phenyl group).

Preferably at least 50 percent of the R¹ groups are methyl. For example, at least 60 percent, at least 70 percent, at least 80 percent, at least 90 percent, at least 95 percent, at least 98 percent, or at least 99 percent of the R¹ groups can be methyl. The remaining R¹ groups can be selected from an alkyl having at least two carbon atoms, haloalkyl, aralkyl, alkenyl, aryl, or aryl substituted with an alkyl, alkoxy, or halo.

Each Y in Formula I is independently an alkylene, aralkylene, or a combination thereof. Suitable alkylene groups typically have up to 10 carbon atoms, up to 8 carbon atoms, up to 6 carbon atoms, or up to 4 carbon atoms. Exemplary alkylene groups include methylene, ethylene, propylene, butylene, and the like. Suitable aralkylene groups usually have an arylene group having 6 to 12 carbon atoms bonded to an alkylene group having 1 to 10 carbon atoms. In some exemplary aralkylene groups, the arylene portion is phenylene. That is, the divalent aralkylene group is phenylene-alkylene where the phenylene is bonded to an alkylene having 1 to 10, 1 to 8, 1 to 6, or 1 to 4 carbon atoms. As used herein with reference to group Y, “a combination thereof” refers to a combination of two or more groups selected from an alkylene and aralkylene group. A combination can be, for example, a single aralkylene bonded to a single alkylene (e.g., alkylene-arylene-alkylene). In one exemplary alkylene-arylene-alkylene combination, the arylene is phenylene and each alkylene has 1 to 10, 1 to 6, or 1 to 4 carbon atoms.

Each subscript n in Formula I is independently an integer of 40 to 1500. For example, subscript n can be an integer up to 1000, up to 500, up to 400, up to 300, up to 200, up to 100, up to 80, or up to 60. The value of n is often at least 40, at least 45, at least 50, or at least 55. For example, subscript n can be in the range of 40 to 1000, 40 to 500, 50 to 500, 50 to 400, 50 to 300, 50 to 200, 50 to 100, 50 to 80, or 50 to 60.

The subscript p is an integer of 1 to 10. For example, the value of p is often an integer up to 9, up to 8, up to 7, up to 6, up to 5, up to 4, up to 3, or up to 2. The value of p can be in the range of 1 to 8, 1 to 6, or 1 to 4.

Group G in Formula I is a residual unit that is equal to a diamine compound of formula R³HN-G-NHR³ minus the two amino groups (i.e., —NHR³ groups). Group R³ is hydrogen or alkyl (e.g., an alkyl having 1 to 10, 1 to 6, or 1 to 4 carbon atoms) or R³ taken together with G and with the nitrogen to which they are both attached may form a heterocyclic group (e.g., R³HN-G-NHR³ is piperazine). The diamine can have primary or secondary amino groups. In most embodiments, R³ is hydrogen or an alkyl. In many embodiments, both of the amino groups of the diamine are primary amino groups (i.e., both R³ groups are hydrogen) and the diamine is of formula H₂N-G-NH₂.

In some embodiments, G is an alkylene, heteroalkylene, polydiorganosiloxane, arylene, aralkylene, or a combination thereof. Suitable alkylenes often have 2 to 10, 2 to 6, or 2 to 4 carbon atoms. Exemplary alkylene groups include ethylene, propylene, butylene, and the like. Suitable heteroalkylenes are often polyoxyalkylenes such as polyoxyethylene having at least 2 ethylene units, polyoxypropylene having at least 2 propylene units, polyoxybutylene or copolymers thereof. Suitable polydiorganosiloxanes include the polydiorganosiloxane diamines of Formula III, which are described below, minus the two amino groups. Exemplary polydiorganosiloxanes include, but are not limited to, polydimethylsiloxanes with alkylene Y groups. Suitable aralkylene groups usually contain an arylene group having 6 to 12 carbon atoms bonded to an alkylene group having 1 to 10 carbon atoms. Some exemplary aralkylene groups are phenylene-alkylene where the phenylene is bonded to an alkylene having 1 to 10 carbon atoms, 1 to 8 carbon atoms, 1 to 6 carbon atoms, or 1 to 4 carbon atoms. As used herein with reference to group G, “a combination thereof” refers to a combination of two or more groups selected from an alkylene, heteroalkylene, polydiorganosiloxane, arylene, and aralkylene. A combination can be, for example, an aralkylene bonded to an alkylene (e.g., alkylene-arylene-alkylene). In one exemplary alkylene-arylene-alkylene combination, the arylene is phenylene and each alkylene has 1 to 10, 1 to 6, or 1 to 4 carbon atoms.

The polydiorganosiloxane polyoxamide tends to be free of groups having a formula —R^{a}—(CO)—NH— where R^{a} is an alkylene. All of the carbonylamino groups along the backbone of the copolymeric material are part of an oxalylamino group (i.e., the —(CO)—(CO)—NH— group). That is, any carbonyl group along the backbone of the copolymeric material is bonded to another carbonyl group and is part of an oxalyl group. More specifically, the polydiorganosiloxane polyoxamide has a plurality of aminoxalylamino groups.

The polydiorganosiloxane polyoxamide is a linear, block copolymer and can be an elastomeric material. Unlike many of the known polydiorganosiloxane polyamides that are generally formulated as brittle solids or hard plastics, the polydiorganosiloxane polyoxamides can be formulated to include greater than 50 weight percent polydiorganosiloxane segments based on the weight of the copolymer. The weight percent of the diorganosiloxane in the polydiorganosiloxane polyoxamides can be increased by using higher molecular weight polydiorganosiloxanes segments to provide greater than 60 weight percent, greater than 70 weight percent, greater than 80 weight percent, greater than 90 weight percent, greater than 95 weight percent, or greater than 98 weight percent of the polydiorganosiloxane segments in the polydiorganosiloxane polyoxamides. Higher amounts of the polydiorganosiloxane can be used to prepare elastomeric materials with lower modulus while maintaining reasonable strength.

The polydiorganosiloxane polyoxamide copolymers have many of the desirable features of polysiloxanes such as low glass transition temperatures, thermal and oxidative stability, resistance to ultraviolet radiation, low surface energy and hydrophobicity, and high permeability to many gases. Additionally, the copolymers exhibit good to excellent mechanical strength.

The copolymeric material of Formula I can be optically clear. As used herein, the term “optically clear” refers to a material that is clear to the human eye. An optically clear copolymeric material often has a luminous transmission of at least about 90 percent, a haze of less than about 2 percent, and opacity of less than about 1 percent in the 400 to 700 nm wavelength range. Both the luminous transmission and the haze can be determined using, for example, the method of ASTM-D 1003-95.

The linear block copolymers having repeat units of Formula I can be prepared, for example by reaction of at least one polydiorganosiloxane-containing precursor with at least one diamine as described in U.S. Pat. No. 7,371,464; incorporated herein by reference.

The diamines are sometimes classified as organic diamines or polydiorganosiloxane diamines with the organic diamines including, for example, those selected from alkylene diamines, heteroalkylene diamines (such as polyoxyalkylene diamines), arylene diamines, aralkylene diamines, or alkylene-aralkylene diamines. The diamine has only two amino groups so that the resulting polydiorganosiloxane polyoxamides are linear block copolymers that are often elastomeric, hot melt processable (e.g., the copolymers can be processed at elevated temperatures such as up to 250° C. or higher without apparent degradation of the composition), and soluble in some common organic solvents. The some embodiments, the diamine is free of a polyamine having more than two primary or secondary amino groups. Tertiary amines that do not react with the polydiorganosiloxane-containing precursor of can also be present. Additionally, the diamines utilized in the reaction are free of any carbonylamino group. That is, the diamine is not an amide.

Preferred alkylene diamines (i.e., G is a alkylene) include, but are not limited to, ethylene diamine, propylene diamine, butylene diamine, hexamethylene diamine, 2-methylpentamethylene 1,5-diamine (i.e., commercially available from DuPont, Wilmington, Del. under the trade designation DYTEK A), 1,3-pentane diamine (commercially available from DuPont under the trade designation DYTEK EP), 1,4-cyclohexane diamine, 1,2-cyclohexane diamine (commercially available from DuPont under the trade designation DHC-99), 4,4′-bis(aminocyclohexyl)methane, and 3-aminomethyl-3,5,5-trimethylcyclohexylamine.

The polydiorganosiloxane polyoxamide copolymer can be produced using a plurality of polydiorganosiloxane precursors, a plurality of diamines, or a combination thereof. A plurality of precursors having different average molecular weights can be combined under reaction conditions with a single diamine or with multiple diamines. For example, the precursor of may include a mixture of materials with different values of n, different values of p, or different values of both n and p. The multiple diamines can include, for example, a first diamine that is an organic diamine and a second diamine that is a polydiorganosiloxane diamine. Likewise, a single precursor can be combined under reaction conditions with multiple diamines.

Any suitable reactor or process can be used to prepare the polydiorganosiloxane polyamide copolymer material. The reaction can be conducted using a batch process, semi-batch process, or a continuous process. Exemplary batch processes can be conducted in a reaction vessel equipped with a mechanical stirrer such as a Brabender mixer, provided the product of the reaction is in a molten state has a sufficiently low viscosity to be drained from the reactor. Exemplary semi-batch process can be conducted in a continuously stirred tube, tank, or fluidized bed. Exemplary continuous processes can be conducted in a single screw or twin screw extruder such as a wiped surface counter-rotating or co-rotating twin screw extruder.

The polydiorganosiloxane-containing precursor can be prepared by any known method. In some embodiments, this precursor is prepared according to the following reaction scheme, as described in previously cited U.S. Pat. No. 7,371,464.

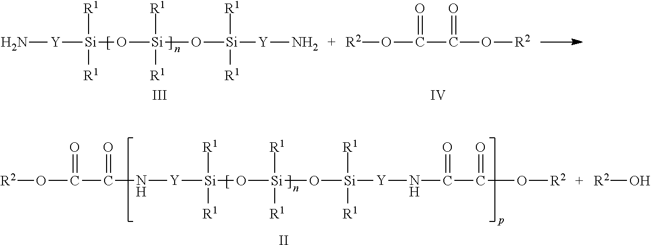

The polydiorganosiloxane diamine can be prepared by any known method and can have any suitable molecular weight. In favored embodiments, the average molecular weight is at least 10,000 g/mole and typically no greater than 150,000 g/mole.

Suitable polydiorganosiloxane diamines and methods of making the polydiorganosiloxane diamines are described, for example, in U.S. Pat. No. 5,214,119 (Leir et al.), U.S. Pat. No. 5,461,134 (Leir et al.), U.S. Pat. No. 5,512,650 (Leir et al.), and U.S. Pat. No. 7,371,464 (Sherman et al.), incorporated herein by reference in their entirety. Some polydiorganosiloxane diamines are commercially available, for example, from Shin Etsu Silicones of America, Inc., Torrance, Calif. and from Gelest Inc., Morrisville, Pa.

Additives such as silicate tackifying resins can optionally be added to the polydiorganosiloxane polyoxamide copolymer to reduce the viscosity or improve adhesion to an adjacent layer in a composite construction.

Suitable silicate tackifying resins include those resins composed of the following structural units M (i.e., monovalent R′₃SiO_{1/2} units), D (i.e., divalent R′₂SiO_{2/2} units), T (i.e., trivalent R′SiO_{3/2} units), and Q (i.e., quaternary SiO_{4/2} units), and combinations thereof. Typical exemplary silicate resins include MQ silicate tackifying resins, MQD silicate tackifying resins, and MQT silicate tackifying resins. These silicate tackifying resins usually have a number average molecular weight in the range of 100 to 50,000 or in the range of 500 to 15,000 and generally have methyl R′ groups.

MQ silicate tackifying resins are copolymeric resins having R′₃SiO_{1/2} units (“M” units) and SiO_{4/2} units (“Q” units), where the M units are bonded to the Q units, each of which is bonded to at least one other Q unit. Some of the SiO_{4/2} units (“Q” units) are bonded to hydroxyl radicals resulting in HOSiO_{3/2} units (“T^{OH}” units), thereby accounting for the silicon-bonded hydroxyl content of the silicate tackifying resin, and some are bonded only to other SiO_{4/2} units.

Such resins are described in, for example, Encyclopedia of Polymer Science and Engineering, vol. 15, John Wiley & Sons, New York, (1989), pp. 265-270, and U.S. Pat. No. 2,676,182 (Daudt et al.), U.S. Pat. No. 3,627,851 (Brady), U.S. Pat. No. 3,772,247 (Flannigan), and U.S. Pat. No. 5,248,739 (Schmidt et al.). Other examples are disclosed in U.S. Pat. No. 5,082,706 (Tangney). The above-described resins are generally prepared in solvent. Dried or solventless, M silicone tackifying resins can be prepared, as described in U.S. Pat. No. 5,319,040 (Wengrovius et al.), U.S. Pat. No. 5,302,685 (Tsumura et al.), and U.S. Pat. No. 4,935,484 (Wolfgruber et al.).

Certain MQ silicate tackifying resins can be prepared by the silica hydrosol capping process described in U.S. Pat. No. 2,676,182 (Daudt et al.) as modified according to U.S. Pat. No. 3,627,851 (Brady), and U.S. Pat. No. 3,772,247 (Flannigan). These modified processes often include limiting the concentration of the sodium silicate solution, and/or the silicon-to-sodium ratio in the sodium silicate, and/or the time before capping the neutralized sodium silicate solution to generally lower values than those disclosed by Daudt et al. The neutralized silica hydrosol is often stabilized with an alcohol, such as 2-propanol, and capped with R₃SiO_{1/2} siloxane units as soon as possible after being neutralized. The level of silicon bonded hydroxyl groups (i.e., silanol) on the MQ resin may be reduced to no greater than 1.5 weight percent, no greater than 1.2 weight percent, no greater than 1.0 weight percent, or no greater than 0.8 weight percent based on the weight of the silicate tackifying resin. This may be accomplished, for example, by reacting hexamethyldisilazane with the silicate tackifying resin. Such a reaction may be catalyzed, for example, with trifluoroacetic acid. Alternatively, trimethylchlorosilane or trimethylsilylacetamide may be reacted with the silicate tackifying resin, a catalyst not being necessary in this case.

MQD silicone tackifying resins are terpolymers having R′₃SiO_{1/2} units (“M” units), SiO_{4/2} units (“Q” units), and R′₂SiO_{2/2} units (“D” units) such as are taught in U.S. Pat. No. 2,736,721 (Dexter). In MQD silicone tackifying resins, some of the methyl R′ groups of the R′₂SiO_{2/2} units (“D” units) can be replaced with vinyl (CH₂═CH—) groups (“D^{Vi}” units).

MQT silicate tackifying resins are terpolymers having R′₃SiO_{1/2} units, SiO_{4/2} units and R′SiO_{3/2} units (“T” units) such as are taught in U.S. Pat. No. 5,110,890 (Butler) and Japanese Kokai HE 2-36234.

Suitable silicate tackifying resins are commercially available from sources such as Dow Corning, Midland, Mich., General Electric Silicones Waterford, N.Y. and Rhodia Silicones, Rock Hill, S.C. Examples of particularly useful MQ silicate tackifying resins include those available under the trade designations SR-545 and SR-1000, both of which are commercially available from GE Silicones, Waterford, N.Y. Blends of two or more silicate resins can be utilized.

The polydiorganosiloxane polyamide compositions typically contain no greater than 15, 10, or 5 weight percent silicate tackifying resin based on the combined weight of polydiorganosiloxane polyamide and silicate tackifying resin. In some embodiments, the polydiorganosiloxane polyamide compositions are free of silicate tackifying resin.

In some embodiments, multi-component fibers are described. The multi-component fibers comprise at least one polydiorganosiloxane polyamide and at least one second polymer (inclusive of copolymers) that is not a polydiorganosiloxane polymer. These different components can be in the form of two or more layered fibers, sheath-core fiber arrangements, fibers with multiple radial segments (e.g. wherein a cross-section of the fiber has a pie arrangement of alternating polydiorganosiloxane polyamide and second melt processable polymer) or in “island in the sea” type fiber structures. At least one layer (e.g. core and/or outer layer) of the multilayered fibers, is present substantially continuously along the fiber length in discrete zones, which zones preferably extend along the entire length of the fibers. In some embodiments, the outer layer is discontinuous along the fiber length.

Regardless of the physical form the multi-component fiber comprises at least about 5, 6, 7, 8, 9, or 10 wt-% and no greater than 25 wt-% polydiorganosiloxane polyamide copolymer based on the total weight of the multi-component fiber.

In a classic sheath-core fiber arrangement, the entire outer layer may consist of the polydiorganosiloxane polyamide composition. Typically, however, a portion of the outer layer comprises the second thermoplastic polymer. Thus, in this embodiment, the polydiorganosiloxane polyamide (e.g. adhesive) comprises less than 100% of the outer surface area. In some embodiments, the polydiorganosiloxane polyamide comprises at least 55%, 60%, 65%, 70%, 75%, 80%, 85%, 90%, 95%, or 100% of the outer surface area of the fibers.

The second polymer(s) are melt processable (typically, thermoplastic) and may or may not have elastomeric properties. By elastomeric properties, it is meant that the polymer can stretch and then return to its original shape (e.g. fiber length) without substantial permanent deformation. In typically embodiments, the second melt processable composition does not have pressure sensitive adhesive properties. Although the second polymer may be miscible with the polydiorganosiloxane polyamide composition, the materials are generally processed such that the outer surface layer is predominantly the polydiorganosiloxane polyamide (e.g. copolymer) composition. The rheological behavior in the melt of the polymers is typically similar in order to facilitate a uniform extrusion.

The second melt processable polymer can provide various improvements. In some embodiments, the second melt processable polymer reduces the cost of the overall web. In other embodiments, the second melt processable polymer can increase the elasticity of the core layer. In yet other embodiments, the second melt processable polymer can improve adhesion or anchoring to another substrate. For example, the inclusion of a (e.g. polyolefin) second melt processable polymer wherein at least a portion is present on the external layer can improve adhesion to other (e.g. polyolefin) materials. The inclusion of the second melt processable polymer improves the liquid barrier properties relative to the polydiorganosiloxane polyamide and second melt processable polymer alone.

The second melt processable polymers or copolymers can be used in various amounts. In favored embodiments, the concentration of a lower cost second melt processable polymers is maximized, while still providing the benefits of the inclusion of the polydiorganosiloxane polyamide (e.g. copolymer) composition. The second melt processable polymer(s) or copolymer(s) is typically present in an amount of at least 75 wt-%, 80 wt-%, 85 wt-%, 90 wt-%, 95 wt-%, based on the total weight of the multi-component fiber or nonwoven web.

Examples of second melt processable polymers or copolymers include, but are not limited to, polyolefins such as polyethylene, polypropylene homopolymer and copolymers, polybutylene, polyhexene, and polyoctene; polystyrenes; polyurethanes; polyesters such as polyethyleneterephthalate; polyamides such as nylon; styrenic block copolymers of the type available under the trade designation KRATON (e.g., styrene/isoprene/styrene, styrene/butadiene/styrene); epoxies; acrylic polymer and copolymers (i.e. polyacrylates); vinyl acetates such as ethylene vinyl acetate; and mixtures thereof.

One illustrative polypropylene polymer is available from Total Petrochemical under the trade designation “TOTAL Polypropylene 3860X”.

In some embodiments, the second melt processable polymer is an elastomer such as polyurethane elastomer or a polyolefin elastomer.

Polyolefin elastomers typically comprise at least 50 wt-% of ethylene, propylene, or combinations thereof. The polyolefin elastomer further comprises one or more C₄-C₂₀ α-olefin, typically in an amount of at least 5, 10, 15 or 20 wt-%. Such polyolefin elastomers generally have a density of at least 0.850, or 0.0855, or 0.860 g/cm³ and no greater than about 0.880 g/cm³, or 0.875 g/cm³, or 0.870 g/cm³. The lower density polymers comprise higher α-olefin contents.

One suitable class of polyolefin elastomers are semicrystalline copolymers and terpolymers of propylene and other α-olefins, such as available from ExxonMobile under the trade designation “Vistamaxx™”. Such elastomers contain greater than 80 wt-% of polypropylene including isotactic polypropylene crystallinity ranging from 5 to 15%.

Other suitable polyolefin elastomers include homogeneous linear ethylene/α-olefin interpolymers are currently available from Mitsui Petrochemical Company under the trade name “Tafmer” and from Exxon Chemical Company under the trade name “Exact”.

Yet another suitable class of polyolefin elastomers includes substantially linear ethylene/α-olefin interpolymers are available from the Dow Chemical Company as Affinity™ polyolefin plastomers as well as Engage™ and Infuse™ polyolefin elastomers. Substantially linear ethylene/α-olefin interpolymers may be prepared in accordance with the techniques described in U.S. Pat. No. 5,272,236 and in U.S. Pat. No. 5,278,272.

Substantially linear C₂-C₃ alkylene/α-olefin interpolymers are homogeneous interpolymers having long chain branching. Due to the presence of such long chain branching, substantially linear C₂-C₃ alkylene/α-olefin interpolymers are further characterized as having a melt flow ratio which may be varied independently of the polydispersity index, and the like, the molecular weight distribution M_{w}/Mₙ. This feature accords substantially linear C₂-C₃ alkylene/α-olefin interpolymers with a high degree of processability despite a narrow molecular weight distribution.

The long chain branches of substantially linear C₂-C₃ alkylene/α-olefin interpolymers have the same comonomer distribution as the interpolymer backbone and can be as long as about the same length as the length of the interpolymer backbone. When a substantially linear C₂-C₃ alkylene/α-olefin interpolymer is employed, such interpolymer will be characterized as having an interpolymer backbone substituted with from 0.01 to 3 long chain branches per 1000 carbons.

Thermoplastic polyurethanes are yet another class of second melt processable elastomers. Such are typically multi-phase block copolymer that are created from a polyol such as a long-chain diol, a short chain-diol chain extender and a diisocyanate. The long chain diol forms a “soft block”; whereas the polymerized short-chain diol forms a “hard block”. The hard block and soft blocks are bonded to each other by means of urethane linkage (i.e. the reaction product of a hydroxyl group and an isocyanate group. Unlike a thermost rubber that comprises non-reversible chemical cross-links, a thermoplastic elastomer comprises physical cross-links of hard block domains that are thermally reversible. Various polyols can be used in the preparation of thermoplastic polyurethane elastomers. Most commonly the polyols are polyester polyols, polyether polyols, and polycaprolactone polyols. Aliphatic isocyanates can be preferred for fibers and nonwovens intended for skin contact.

The second melt processable polymers typically have a melt flow index according to ASTM D-1238 (at the temperature specified in the test method) of at least 10 g/10 min, 20 g/10 min., or 30 g/10 min and no greater than about 125 g/10 min or 100 g/10 min.

In some embodiments, the second melt processable polymer has a Shore A hardness of at least 50, 55, or 60. Further the flexural modulus (ASTM D-790) is typically at least 1000, 1200, or 1400 MPa and in some embodiments no greater than 3000 MPa. Further, the tensile modulus (ASTM D-638) is typically at least 800 or 900 MPa and can range up to 2000 MPa. The elastomers typically have an elongation at break of at least 500, 1000, 1500, or 2000% or greater.

The polydiorganosiloxane polyamide polymer composition and the second melt processable polymer can further include other additives to provide desired properties. For example, dyes and pigments can be added as colorant; electrically and/or thermally conductive compounds can be added to make the composition electrically and/or thermally conductive or antistatic; antioxidants and antimicrobial agents can be added; and ultraviolet light stabilizers and absorbers, such as hindered amine light stabilizers (HALS), can be added to stabilize the composition against ultraviolet degradation and to block certain ultraviolet wavelengths from passing through the article. Other additives include, but are not limited to, adhesion promoters, fillers, tack enhancers (i.e. tackifiers), blowing agents, melt processable diluents such as plasticizers, and flame-retardants.

Melt processes for the preparation of fibers such as spunbond and melt-blown processes are well-known in the art. Although the multi-component fiber can be prepared from any suitable process, the composition is preferably prepared into fibers, particularly microfibers, and nonwoven webs thereof, with a melt-blown process as described in U.S. Pat. Nos. 5,238,733 and 6,083,856 (Joseph et al.); incorporated herein by reference.

Melt-blown processes are particularly preferred because they form autogeneously bonded webs that typically require no further processing to bond the fibers together. The melt-blown processes used in the formation of multilayer microfibers as disclosed in the Joseph et al. patents listed above are particularly suitable for use in making the multilayer microfibers described herein. Such processes use hot (e.g., equal to or about 20° C. to about 30° C. higher than the polymer melt temperature), high-velocity air to draw out and attenuate extruded polymeric material from a die, which will generally solidify after traveling a relatively short distance from the die. Depending on the processing temperature and conditions, the melt-blown fibers thus formed can be unoriented (i.e. lack orientation). The resultant fibers are termed melt-blown fibers and are generally substantially continuous. They form into a coherent web either on the collecting surface or between the exit die orifice and a collecting surface by entanglement of the fibers due in part to the turbulent airstream in which the fibers are entrained.

For example, the Joseph et al. patents describe forming a multi-component melt-blown microfiber web by feeding two separate flow streams of organic polymeric material into a separate splitter or combining manifold. The split or separated flow streams are generally combined immediately prior to the die or die orifice. The separate flow streams are preferably established into melt streams along closely parallel flow paths and combined where they are substantially parallel to each other and the flow path of the resultant combined multilayered flow stream. This multilayered flow stream is then fed into the die and/or die orifices and through the die orifices. Air slots are disposed on either side of a row of the die orifices directing uniform heated air at high velocities at the extruded multi-component melt streams. The hot high velocity air draws and attenuates the extruded polymeric material which solidified after traveling a relatively short distance from the die. Single layer microfibers can be made in an analogous manner with air attenuation using a single extruder, no splitter, and a single port feed die.

The temperature and selection of melt processable material of the separate flowstreams is typically controlled to bring the polydiorganosiloxane (e.g. copolymer) polyamide and second melt processable polymers to sufficiently similar complex viscosities, as previously described.

The solidified or partially solidified fibers form an interlocking network of entangled fibers that are collected as a web. The collecting surface can be a solid or perforated surface in the form of a flat surface or a drum, a moving belt, or the like. If a perforated surface is used, the backside of the collecting surface can be exposed to a vacuum or low-pressure region to assist in the deposition of the fibers. The collector distance is generally about 5, 6, or 7 centimeters (cm) to about 130 cm from the die face. Moving the collector closer to the die face, e.g., about 7 cm to about 30 cm, will result in stronger inter-fiber bonding and a less lofty web.

The size of the polymeric fibers formed depends to a large extent on the velocity and temperature of the attenuating airstream, the orifice diameter, the temperature of the melt stream, and the overall flow rate per orifice. The webs formed can be of any suitable thickness for the desired and intended end use. The thickness of the web is typically at least about 0.10, 0.15, 0.20 or 0.20 mm. For high moisture vapor transmission rates in combination with good liquid barrier properties, the thickness is typically at least 0.30, or 0.35 mm. In typical embodiments, the thickness of the web is no greater than about 3, 2, or 1 mm. The basis weight of the nonwoven web typically ranges from about 5 grams/m² to about 100, 125, 150, 175, or 200 grams/m² microns. In some embodiments, the basis weight is at least 10, 15 or 20 grams/m².

The multi-component fibers described herein can be mixed with other fibers, such as staple fibers. Webs having more than one type of fiber are referred to herein as having commingled constructions. The various types of fibers can be intimately mixed forming a substantially uniform cross-section or they can be in separate layers. The web properties can be varied by the number of different fibers used, the number of layers employed, and the layer arrangement. Other materials, such as surfactants or binders can also be incorporated into the web before, during, or after its collection, such as by the use of a spray jet.

Webs or composite structures including the webs can be further processed after collection or assembly, such as by calendaring or point embossing to increase web strength, provide a patterned surface, or fuse fibers at contact points in a web structure or the like; by orientation to provide increased web strength; by needle punching; heat or molding operations; coating, such as with adhesives to provide a tape structure; or the like.

The nonwoven webs described herein can be used in composite multi-layer structures. The other layers can be supporting webs, nonwoven webs of spun bond, staple, and/or melt-blown fibers, as well as films of elastic, semipermeable, and/or impermeable materials. These other layers can be used for absorbency, surface texture, rigidification, etc. They can be attached to the nonwoven webs of the fibers using conventional techniques such as heat bonding, binders or adhesives, or mechanical engagement such as hydroentanglement or needle punching.

The nonwoven webs described herein can be used as a backing for various articles wherein a combination of high moisture vapor transmission alone or in combination with fluid barrier properties are desired

The nonwoven webs described herein can be used to prepare adhesive articles, such as tapes, including medical grade tapes that adhere to skin, labels, wound dressings, so-called incise drapes that are adhered to the skin of a patient prior to making a surgical incision directly through the drape and the like. In one embodiment, fluids are actively removed from a sealed environment provided by the wound dressing (such as described in US 2010/0318052).

In one embodiment, the nonwovens webs are useful as a conformable backing of a medical or wound dressing.

The nonwoven web suitable for use as a backing substrate typically has an upright moisture vapor transmission rate of at least 1000, or 1500, or 2000, or 2500, or 3000 g/m²/24 hrs. In some embodiments, the upright moisture vapor transmission rate of at least 4000, or 5000, or 6000, or 7000, or 8000 g/m²/24 hrs. In some embodiments, the upright moisture vapor transmission rate is no greater than about 10,000 g/m²/24 hrs. The inverted moisture vapor transmission rate of the backing substrate is typically at least 10,000, or 15,000, or 20,000 g/m²/24 hrs. In some embodiments, the inverted moisture vapor transmission rate is no greater than about 40,000 g/m²/24 hrs.

The wound dressing typically transmits moisture vapor at a rate equal to or greater than human skin. In some embodiments, the adhesive coated backing transmits moisture vapor at a rate of at least 200 or 250 g/m²/24 hrs/37° C./100-10% RH, frequently at least 700 g/m²/24 hrs/37° C./100-10% RH, when the adhesive is in contact with water vapor and not water (i.e. upright MVTR) and most typically at least 2000 g/m²/24 hrs/37° C./100-10% RH when adhesive is in contact with water, using the inverted cup method (such as described in U.S. Pat. No. 4,595,001).

In some favored embodiments, the nonwoven web suitable for use as a backing substrate exhibits good liquid barrier properties in combination with high moisture vapor transmission rates. In some embodiments, the nonwoven web resists water penetration with a constant water pressure of 20 mbar for at least 5, 6, 7, 8, 9, or 10 minutes as measured according to the “Barrier Performance” described in greater detail in the forthcoming examples. In some embodiments, the nonwoven web resists water penetration for at least 15, 20, 25, 30, 35, or 40 minutes ranging up to about 1, 1.5 or 2 hours. Water penetration resistant is typically a good test fluid for other fluids including bodily fluids.

The backing nonwoven as described herein is conformable to anatomical surfaces. As such, when the backing substrate is applied to an anatomical surface, it conforms to the surface even when the surface is moved. The backing substrate may also be conformable to animal anatomical joints. When the joint is flexed and then returned to its unflexed position, the backing substrate may stretch to accommodate the flexion of the joint, but is resilient enough to continue to conform to the joint when the joint is returned to its unflexed condition. A description of this characteristic of backing substrates can be found in issued U.S. Pat. Nos. 5,088,483 and 5,160,315.

Pressure sensitive adhesives for wound dressings include those based on acrylates, polyurethanes, KRATON and other block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including for example an antimicrobial agent. In some embodiments, the pressure sensitive adhesive has a relatively high moisture vapor transmission rate to allow for moisture evaporation, as previously described with regard to the backing substrate. This can be achieved by pattern coating, etc. as known in the art.

Specific adhesives that are commonly applied to the skin include acrylate copolymers such as described in U.S. Pat. No. RE 24,906, particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Release liners typically protect the pressure sensitive adhesive used to attach the dressings to the patient and in some embodiments create a sealed cavity. Release liners that may be suitable for use in the medical dressing can be made of supercalendered kraft paper, glassine paper, polyethylene, polypropylene, polyester or composites of any of these materials. The liners are coated with release agents such as fluorochemicals or silicones such as described in US 20120/0318052.

An absorbent material may also be used in conjunction with the medical dressings described herein. The absorbent materials can be manufactured of any of a variety of materials including, but not limited to, woven or nonwoven materials such as cotton or rayon. Absorbent pad is useful for containing a number of substances, optionally including antimicrobial agents, drugs for transdermal drug delivery, chemical indicators to monitor hormones or other substances in a patient, etc.

The absorbent may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof.

Absorbent materials may also be chosen from other synthetic and natural hydrophilic materials including polymer gels and foams. The foams can be open cell polyurethane, closed cell polyurethane.

The medical dressings can further comprise valves, barrier elements, septum elements, at least one of a number of active ingredients etc. as described in US 2010/0318052; incorporated herein by reference.

In some instances, the backing nonwoven as described herein is used in the medical dressings may be so flexible and supple such that when a release liner is removed from the backing substrate, the backing substrate may tend to fold and adhere to itself, interfering with the smooth, aseptic application of the dressing to a patient's skin.

Carrier materials such as frames, handles, stiffening strips, etc. as known in the art are one way to prevent the backing substrate from folding and adhering to itself. Carrier materials can include, but are not limited to, ethylene vinyl acetate copolymer or ethylene acrylic acid coated papers and polyester films.

The carrier material can be heat seal-bonded to the backing substrate. In such embodiments, the low adhesion coating is compatible with the heat seal bond between the carrier and the backing substrate and also retains its low coefficient of friction characteristics after heat sealing. Further the low adhesion coating can also reduce the heat seal bond strength between the backing and the carrier such that the carrier is retained, yet can be easily removed during use.

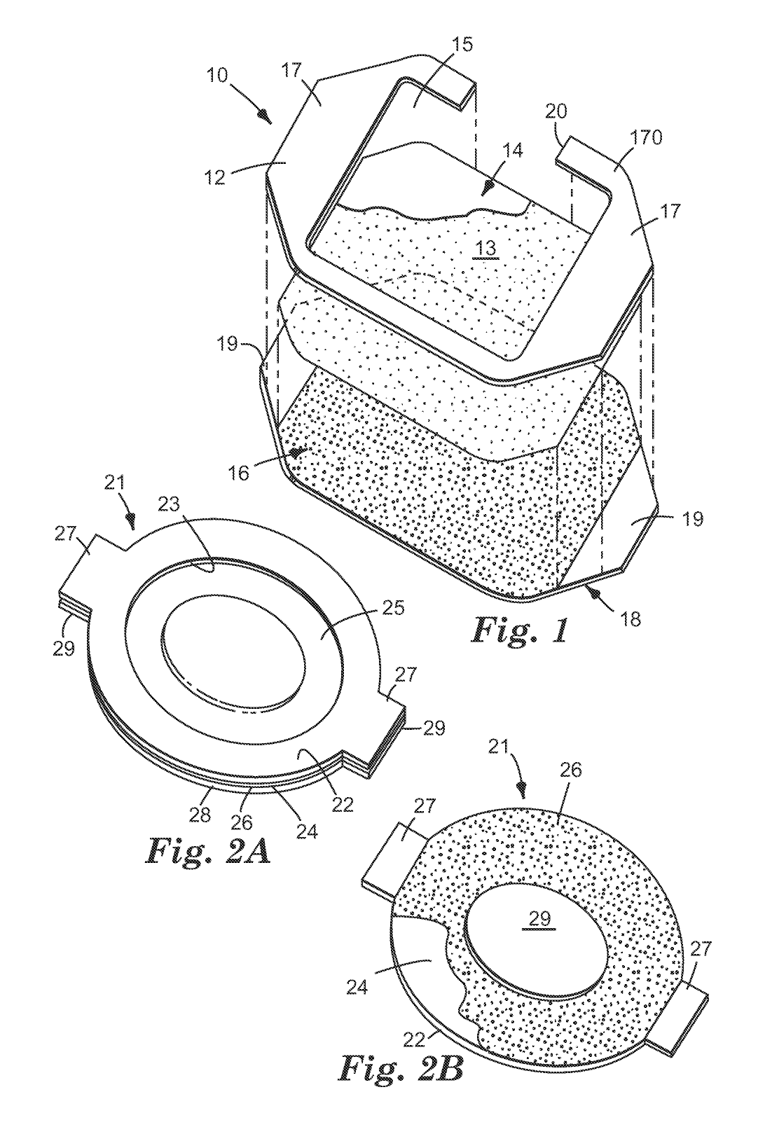

One illustrative medical dressing is depicted in FIG. 1 and described in U.S. Pat. No. 5,531,855; incorporated herein by reference. The adhesive composite dressing 10 comprises a (e.g. conformable) backing substrate 14; a low adhesion coating 13 on a top face of the backing substrate 14; a carrier 170 attached to the top face of the backing substrate 14 over the low adhesion coating 13; a pressure-sensitive adhesive 16 on a bottom face of the backing substrate 14; and a liner 18 attached to the exposed surface of pressure-sensitive adhesive 16.

The carrier 170 is typically attached to backing substrate 14 through low adhesion coating 13 with a heat seal bond. In one embodiment, a (e.g. rectangular) window portion cut in the carrier 170 is removed creating a frame 12 and a window 15 exposing a portion of the top face of the backing substrate 14. Carrier (e.g. frame) 12 provides rigidity to the backing substrate 14 after liner 18 is removed. However, the removal of the window portion of the carrier material 170 is optional. In either embodiment, the low adhesion coating 13 becomes incorporated into and does not impair the formation of a heat seal bond between the carrier (e.g. frame) 12 and backing substrate 14. Further, the heat seal bond comprises materials from all three layers—i.e. the carrier material, the low adhesion coating and the backing substrate.

Liner 18 and carrier (e.g. frame) 12 can both include tabs 17 and 19 that extend beyond the perimeter of backing substrate 14 to provide a means of applying the dressing without contacting the adhesive 16.

The heat seal bond between the carrier 170 and the backing substrate 14 is stronger than the bond between the adhesive 16 and the liner 18. That difference ensures that the backing substrate 14 remains attached to the frame 12 when liner 18 is removed from the adhesive composite dressing 10.

The dressing 10, having frame 12 that includes opening 20 such that the frame 12 does not extend completely around the perimeter of the backing substrate 14 can be placed over catheters or other devices while still attached to the frame 12 to increase the ease of handling of backing substrate 14.

In use, liner 18 is first removed from the adhesive composite dressing 10 leaving the frame 12/backing substrate 14/pressure-sensitive adhesive 16 intact. The user can then manipulate the adhesive composite dressing 10 using tabs 17 on the frame 12 while viewing the area to which the dressing 10 will be attached through window 15, as the (e.g. transparent or translucent) backing substrate 14.

FIGS. 2A and 2B, depict an alternate embodiment of a medical dressing 21. As shown, the medical dressing 21 is an adhesive composite comprising a frame 22, a backing substrate 24, adhesive 26 and a liner 28. The backing substrate comprises the block copolymer coating described herein on the surface between the backing and the frame. Liner 28 may have opposing tabs 27 for handling, and frame 22 also includes tabs 27 for handling.

Medical dressing 21 also includes an open area or window 25 in frame 22 which exposes a portion of the top surface of backing 24. Frame 22 extends around the entire perimeter of backing 24 and includes a control depth die cut 23 to facilitate removal of frame 22 from backing 24 after the dressing 21 has been applied to a patient.

FIG. 2B is a bottom view of medical dressing 1 with liner 28 removed to expose the adhesive layer 26 and absorbent pad 29 disposed proximate the center of the dressing 21. Absorbent pad 29 can be manufactured of a number of materials including, but not limited to, woven or nonwoven cotton or rayon. Absorbent pad 29 is useful for containing a number of substances, including antimicrobial agents, drugs for transdermal drug delivery, chemical indicators to monitor hormones or other substances in a patient, etc. Furthermore, although absorbent pad 29 is shown as centered on dressing 21, it can take any appropriate shape and/or can be located off-center on the dressing 21 as desired.

Removal of the frame material 22 from the window area 25 of dressing 21 can be advantageous. Pad 29 tends to deform the backing 24 and cause delamination between the frame material 22 in window 25 if that material is still present when pad 29 is placed on dressing 21.

This invention is further illustrated by the following examples which are not intended to be limiting in scope. Unless indicated otherwise, the molecular weights refer to number average molecular weights. All parts, percentages and ratios are by weight unless otherwise specified.

In some embodiments the polydiorganosiloxane polyamide copolymer may have incorporated therein or thereon one or more suitable antimicrobial agents. The use of an antimicrobial agent can be particularly useful for topical applications such as wound dressings, incise drapes, IV dressings, first aid dressings, medical tapes, wound contact layers, and the like. The antimicrobial agent includes an antimicrobial lipid, a phenolic antiseptic, a cationic antiseptic, iodine and/or an iodophor, an antimicrobial natural oil, or combinations thereof. In addition to or in place of the antimicrobial in or on the fiber comprising the polydiorganosiloxane polyamide PSA, the medical article may have antimicrobial delivered from one or more other components such as a nonwoven or foam layer, the backing, a thin film contact layer and the like.

In certain embodiments, the antimicrobial lipid is selected from the group consisting of a (C7-C14)saturated fatty acid ester of a polyhydric alcohol, a (C8-C22)unsaturated fatty acid ester of a polyhydric alcohol, a (C7-C14)saturated fatty ether of a polyhydric alcohol, a (C8-C22)unsaturated fatty ether of a polyhydric alcohol, a (C7-C14)fatty alcohol ester (preferably a monoester) of a (C2-C8)hydroxycarboxylic acid (preferably a (C8-C12)fatty alcohol ester (preferably a monoester) of a (C2-C8)hydroxycarboxylic acid), a (C8-C22)mono- or poly-unsaturated fatty alcohol ester (preferably a monoester) of a (C2-C8)hydroxycarboxylic acid, an alkoxylated derivative of any of the foregoing having a free hydroxyl group, and combinations thereof; wherein the alkoxylated derivative has less than 5 moles of alkoxide per mole of polyhydric alcohol or hydroxycarboxylic acid; with the proviso that for polyhydric alcohols other than sucrose, the esters comprise monoesters and the ethers comprise monoethers, and for sucrose the esters comprise monoesters, diesters, or combinations thereof, and the ethers comprise monoethers.

In certain embodiments, the antimicrobial lipid is selected from the group consisting of a (C8-C12)saturated fatty acid ester of a polyhydric alcohol, a (C12-C22)unsaturated fatty acid ester of a polyhydric alcohol, a (C8-C12)saturated fatty ether of a polyhydric alcohol, a (C12-C22)unsaturated fatty ether of a polyhydric alcohol), an alkoxylated derivative of any of the foregoing, and combinations thereof; wherein the alkoxylated derivative has less than 5 moles of alkoxide per mole of polyhydric alcohol; with the proviso that for polyhydric alcohols other than sucrose, the esters comprise monoesters and the ethers comprise monoethers, and for sucrose the esters comprise monoesters, diesters, or combinations thereof, and the ethers comprise monoethers.

In certain embodiments, the antimicrobial component includes a phenolic antiseptic. In certain embodiments, the phenolic antiseptic is selected from the group consisting of diphenyl ethers, phenolics, bisphenolics, resorcinols, anilides, and combinations thereof. In certain embodiments, the phenolic antiseptic comprises triclosan.

In certain embodiments, the antimicrobial component includes a cationic antiseptic. In certain embodiments, the cationic antiseptic is selected from the group consisting of biguanides, bisbiguanides, polymeric biguanides, including but not limited to chlorhexidine salts and polyhexamethylene biguanide salts, polymeric quaternary ammonium compounds, silver and its complexes, small molecule quaternary ammonium compounds comprising a quaternary ammonium or protonated tertiary or secondary amine and at least one alkyl group of at least 6 carbon atoms such as benzethonium chloride, methylbenzethonium chloride, benzalkonium chloride, cetylpyridinium chloride, cetyltrimethylammonium bromide, octenidine, and the like, and combinations thereof.

In certain embodiments the cationic antimicrobial component includes silver and copper. Silver is also known to be an effective antiseptic and has been used in creams to treat wounds and other topical infections. The active form of silver is the ion Ag+ which may be delivered from a variety of well known silver salts and complexes including silver zeolites; inorganic silver salts such as silver nitrate, silver chloride, silver sulfate, silver thiosulfate; silver alkyl, aryl, and aralkyl carboxylates (e.g. carboxylate anions having less than about 8 carbon atoms such as the acetate, lactate, salicylate, and gluconate salts); silver oxide, colloidal silver, nanocrystalline silver, silver zeolites, silver coated microspheres, silver complexed with various polymers as well as silver delivered from dendrimers as described in U.S. Pat. Nos. 6,579,906 and 6,224,898; and silver antimicrobial complexes such as silver sufadiazine. The silver may optionally complexed with primary, secondary, tertiary, and quaternary amines as well as polymeric forms thereofs, and silver protein complexes.

In certain embodiments, the antimicrobial component includes iodine and/or an iodophor. In certain embodiments, the iodophor is povidone-iodine or a polyethyleneglycol-iodine or derivatized polyethylene glycol-iodine complex such as a polyethyoxylated alkyl or alkaryl alcohol-iodine complex. As used herein an iodine complex may include complexes with molecular iodine but are more often complexes with triiodide.

In certain embodiments, the antimicrobial component includes an antimicrobial natural oil. The natural oil antiseptics includes oils and oil extracts from plants such as Tea Tree oil, grape fruit seed extract, Aspidium extract (phloro, lucinol containing extract); barberry extract (berberine chloride); bay sweet extract; bayberry bark extract (myricitrin); cade oil; CAE (available from Ajinomoto, located in Teaneck, N.J.); cajeput oil; caraway oil; cascarilla bark (sold under the tradename ESSENTIAL OIL); cedarleaf oil; chamomille; cinnamon oil; citronella oil; clove oil; German chamomile oil; giant knotweed; lemon balm oil; lemon grass; olive leaf extract (available from Bio Botanica); parsley; patchouly oil; peony root; pine needle oil; PLANSERVATIVE (available from Campo Research); rose geranium oil; rosemary; sage, and the like, as well as mixtures thereof.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

Materials utilized in the sample preparation are shown in Table 1.

TABLE 1
Components

| Component | Description | Source |
|---|---|---|
| PP | Polypropylene 3860X | Total Petrochemical, |
| | | Houston, TX |
| TPU | Irogran ® PS440-200 | Huntsman International |
| | Thermoplastic | LLC, Woodlands, TX |
| | Polyurethane elastomer | |
| VM | Vistamaxx 2125 Grade | ExxonMobile Chemical |
| | Polymer | Company, Houston, TX |

The Silicone polyoxamide (SPOx) polymer used is indicated by this structural formula

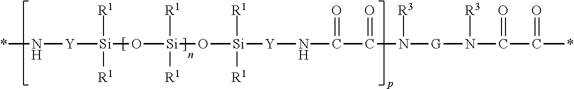

in which Y=propylene, R1=methyl, R3=H, and G=ethylene. It was prepared according to the method of Example 2 in U.S. Pat. No. 7,501,184, with the exception that instead of a 5,000 molecular weight polydimethylsiloxane diamine, a PDMS diamine having an approximate molecular weight of 25,000 g/mol was used. The 25,000 MW silicone diamine was prepared as described in U.S. Pat. No. 6,355,759.

To prepare the silicone polyoxamide, a molar ratio of amine (from ethylene diamine) to ester (from the precursor) of 0.92:1 was used.

Inherent Viscosity (IV) of this Silicone polyoxamide polymer was measured at 30° C., using a Canon-Fenske viscometer (Model No. 50 P296), in a tetrahydrofuran (THF) solution at a concentration of 0.2 g/dL. Inherent viscosities of similar Silicone polyoxamide polymers have been found previously to be essentially independent of concentration in the range of 0.1 to 0.4 g/dL. The inherent viscosity was averaged over 3 runs. The Silicone polyoxamide resin had an IV of 1.6 dl/g.

### Test Methods

### Fiber Diameter

The effective fiber diameter (EFD) was determined according to the method set forth in Davies, C. N., “The Separation of Airborne Dust and Particles,” Institution of Mechanical Engineers, London Proceedings 1B, 1952.

### Upright MVTR

The upright MVTR was measured according to ASTM E96-80 using a modified Payne cup method. A 3.8 cm diameter sample was placed between adhesive-containing surfaces of two foil adhesive rings, each having a 5.1 cm² elliptical opening. The holes of each ring were carefully aligned. Finger pressure was used to form a foil/sample/foil assembly that was flat, wrinkle free, and had no void areas in the exposed sample.

A 120 mL glass jar was filled with approximately 50 g of tap water that contained a couple drops of 0.02% (w/w) aqueous Methylene Blue USP (Basic Blue 9, C.I.52015) solution, unless specifically stated in an example. The jar was fitted with a screw-on cap having a 3.8 cm diameter hole in the center thereof and with a 4.45 cm diameter rubber washer having an approximately 3.6 cm hole in its center The rubber washer was placed on the lip of the jar and foil/sample/foil assembly was placed backing side down on the rubber washer. The lid was then screwed loosely on the jar.

The assembly was placed in a chamber at 40° C. and 20% relative humidity for four hours. At the end of four hours, the cap was tightened inside the chamber so that the sample was level with the cap (no bulging) and the rubber washer was in proper seating position.

The foil sample assembly was removed from the chamber and weighed immediately to the nearest 0.01 gram for an initial dry weight, W1. The assembly was then returned to the chamber for at least 18 hours, the exposure time T1 in hours, after which it was removed and weighed immediately to the nearest 0.01 g for a final dry weight, W2. The MVTR in grams of water vapor transmitted per square meter of sample area per 24 hours can then be calculated using the following formula.

Upright (Dry) MVTR=(W1−W2)*(4.74*104)/T1

### Inverted MVTR

The inverted MVTR was measured using the following test procedure. After obtaining the final “dry” weight, W2, as described for the upright MVTR procedures, the assembly was returned to the chamber for at least 18 additional hours of exposure time, T2, with the jars inverted so that the tap water was in direct contact with the test sample. The sample was then removed from the chamber and weighed to the nearest 0.01 gram for a final wet weight, W3. The inverted wet MVTR in grams of water vapor transmitted per square meter of sample area per 24 hours can then be calculated using the following formula.

Inverted (Wet) MVTR=(W2−W3)*(4.74*104)/T2

### Barrier Performance

European test standard EN20811 was used to measure the resistance to water penetration. Briefly, a 10 cm×10 cm backing sample was clamped to the test head of a Textest Instruments FX3000 (Textest Instruments, Schwerzenbach, Switzerland), and was subjected on one face to a constant water pressure of 20 mbar. The sample was monitored for water penetration. The time until water penetrated in a third location was recorded.

### Example 1

A web was prepared using a melt blowing process similar to that described in Example 1 of U.S. Pat. No. 5,238,733 (Joseph et al). A Brabender conical twin screw extruder, set to 310° C., delivered PP to form the core layer of the fibers, and a Bonnot extruder, also set to 310° C., delivered SPOx resin to form the shell layers of the fibers. The core and shell polymers were combined in a three layer feedblock and extruded through a 10 hole/cm drilled orifice die. The collector was approximately 18 cm from the die. The extruded core/shell fiber was formed into a nonwoven web with a basis weight of 113 g/m² and effective fiber diameter of 7.7 micrometer.

### Examples 2-12

Additional examples were prepared in a similar manner to Example 1 as described in Table 2 such that the outer (e.g. shell layer) comprised SPOx and the core was a (e.g. second) melt processible polymer other than SPOx.

### Comparatives

Webs were prepared with 100% SPOx fibers (Comparative 1) and 100% TPU fibers (Comparative 2). In addition, a commercially available non-woven from First Quality (34 gsm SMS, style SM3397039) was tested (Comparative 3).

TABLE 2
Backing Samples

| | Multi-Component Fiber | | | | | | |
|---|---|---|---|---|---|---|---|
| | Composition | | | | | Web | |
| | SPOx | Second Polymer | | | Fiber | Basis | Web |
| | (wt. | (wt. %) | | | Diameter | Weight | Thickness |
| | %) | PP | TPU | VM | (μm) | (g/m²) | (mm) |
| Example | | | | | | | |
| 1 | 10 | 90 | 0 | 0 | 7.7 | 113 | 0.86 |
| 2 | 10 | 90 | 0 | 0 | 7.8 | 100 | 0.81 |
| 3[a] | 10 | 90 | 0 | 0 | 7.8 | 100 | 0.28 |
| 4 | 20 | 80 | 0 | 0 | 7.8 | 100 | 0.79 |
| 5 | 10 | 45 | 0 | 45 | 10.9 | 100 | 0.51 |
| 6 | 20 | 40 | 0 | 40 | 10.9 | 100 | 0.51 |
| 7 | 20 | 20 | 0 | 60 | 10.9 | 100 | 0.53 |
| 8 | 10 | 23 | 0 | 67 | 10.9 | 100 | 0.43 |
| 9 | 10 | 9 | 0 | 81 | 19.4 | 99 | 0.40 |
| 10 | 20 | 8 | 0 | 72 | 19.4 | 99 | 0.36 |
| 11 | 10 | 0 | 90 | 0 | 12.4 | 102 | 0.38 |
| 12 | 20 | 0 | 80 | 0 | 12.4 | 102 | 0.38 |
| Comparative | | | | | | | |

| | | | | |
|---|---|---|---|---|
| 1 | 100% SPOx | [b] | [b] | [b] |
| 2 | 100% TPU | 12.4 | 102 | 0.38 |
| 3 | First Quality non-woven | [b] | [b] | [b] |
| | web | | | |

[a]= die to collector distance was approximately 8 cm
[b] = not measured

Results are shown in Tables 3.

TABLE 3
Test Results

| | Upright MVTR | Inverted MVTR | Barrier Performance |
|---|---|---|---|
| | (g/m²/24 hours) | (g/m²/24 hours) | (min) |
| Example | | | |
| 1 | 7992 | 19413 | >42 |
| 2 | 8141 | 21350 | >42 |
| 3 | 1888 | leaked | 0 |
| 4 | 8025 | 18849 | >42 |
| 5 | 8640 | 23624 | 12.5 |
| 6 | 8307 | 18231 | >42 |
| 7 | 8540 | 19681 | >42 |
| 8 | 8581 | 18657 | 14 |
| 9 | 8448 | 18883 | 1 |
| 10 | 8115 | 26255 | 2 |
| 11 | 8307 | 27855 | 10 |
| 12 | 8107 | 26358 | >42 |
| Comparative | | | |
| 1 | Sample tore | Sample tore | Sample tore |
| 2 | 8756 | 27505 | 0 |
| 3 | 8347 | 33278 | 4.1 |

1. A multi-component fiber comprising a core and outer layer, wherein at least a portion of the outer layer comprises a first melt processable composition comprising a polydiorganosiloxane polyamide copolymer and the core comprises a second melt processable composition that does not comprise a polydiorganosiloxane polymer and wherein the multi-component fiber comprises 5 to 25 wt-% polydiorganosiloxane polyamide copolymer and the first melt processable composition is not a pressure sensitive adhesive.

2. The multi-component fiber of claim 1 wherein the polydiorganosiloxane polyoxamide copolymer comprises at least two repeat units of Formula I: 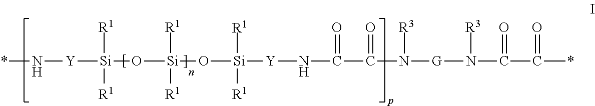
wherein
each R¹ is independently an alkyl, haloalkyl, aralkyl, alkenyl, aryl, or aryl substituted with an alkyl, alkoxy, or halo, wherein at least 50 percent of the R¹ groups are methyl;
each Y is independently an alkylene, aralkylene, or a combination thereof;
G is a divalent residue equal to a diamine of formula R³HN-G-NHR³ minus the two —NHR³ groups;
R³ is hydrogen or alkyl or R³ taken together with G and with the nitrogen to which they are both attached forms a heterocyclic group;
n is independently an integer of 40 to 1500; and
p is an integer of 1 to 10; and
an asterisk (*) indicates a site of attachment of the repeat unit to another group in the copolymer.

3-7. (canceled)

8. The multi-component fiber of claim 1 wherein the first melt processable composition is not a pressure sensitive adhesive.

9. The multi-component fiber of claim 1 wherein the second melt processable composition is selected from a polyolefin polymer, a polyolefin elastomer, a polyurethane elastomer, or a mixture thereof.

10. The multi-component fiber of claim 9 wherein the second melt processable composition is polyolefin and the multi-component fiber comprises 75 wt-% to 95 wt-% polyolefin.

11. The multi-component fiber of claim 10 wherein the polyolefin is a polypropylene homopolymer or copolymer.

12. The multi-component fiber of claim 9 wherein the second melt processable composition is a blend of polyolefin polymers.

13. The multi-component fiber of claim 12 wherein the polyolefin elastomer is present is the blend in an amount ranging from 5 to 65 wt-%.

14. A nonwoven web comprising the multi-component fiber of claim 1.

15. The nonwoven web of claim 14 wherein the nonwoven web has a thickness of at least 0.10 mm.

16. The nonwoven web of claim 14 wherein the multi-component fiber has an average fiber diameter ranging from 5 to 50 micrometers.

17. The nonwoven web of claim 16 wherein the multi-component fiber has an average fiber diameter no greater than 15 micrometers.

18. The nonwoven web of claim 14 wherein the nonwoven web has a basis weight ranging from 25 to 200 g/m².

19. The nonwoven web of claim 14 wherein the nonwoven web has an Upright MVTR of at least 1000, 3000, or 5000 g/m²/24 h.

20. The nonwoven web of claim 14 wherein the nonwoven web has an Inverted MVTR of at least 10,000 g/m²/24 h.

21. The nonwoven web of claim 14 wherein the nonwoven web has resistance to water penetration of at least 5 or 10 min according to EN20811.

22. The nonwoven web of claim 14 wherein the nonwoven web further comprises an antimicrobial.

23. A nonwoven web wherein the web has an inverted MVTR of at least 10,000 g/m²/24 h and a resistance to water penetration of at least 10 min according to EN20811 for a water pressure of 20 mbar wherein the nonwoven web comprises a multi-component fiber comprising a core and outer layer, wherein at least a portion of the outer layer comprises a first melt processable composition comprising a polydiorganosiloxane polyamide copolymer and the core comprises a second melt processable composition that does not comprise a polydiorganosiloxane polymer.

24. (canceled)

25. A medical article comprising a backing, the backing comprising the nonwoven web according to claim 14.

26. The medical article of claim 25 wherein the article is selected from the group consisting of tapes, wound dressings, and incise drapes.

26-30. (canceled)
